(19) United States
(12) Patent Application Publication
(10) Pub. No.: US 2010/0224187 A1
(43) Pub. Date: Sep. 9, 2010

(54) APPARATUS AND METHODS FACILITATING ATRAUMATIC INTUBATION

(76) Inventor: Thomas Maxwell Dalton, Wilmington, NC (US)

Correspondence Address: TILLMAN WRIGHT, PLLC, PO BOX 49309, CHARLOTTE, NC, 28277-0076, US

(21) Appl. No.: 12/717,939

(22) Filed: Mar. 4, 2010

Related U.S. Application Data
(60) Provisional application No. 61/157,547, filed on Mar. 4, 2009.

(51) Int. Cl. A61M 16/00

(52) U.S. Cl. 128/200.26

(57) ABSTRACT

A controllable intubating stylet (CIS) for use by anesthesia and other health care providers is used in conjunction with a video laryngoscope and endotracheal tube in order to achieve intubation of the trachea for general anesthesia as well as other medical conditions. The video laryngoscope is used to visualize the tracheal opening. The CIS is inserted into an endotracheal tube and directed into the trachea. The endotracheal tube then is maneuvered over the stylet and into the trachea, and thereafter, the CIS is removed. The patient can then be oxygenated and ventilated by way of the endotracheal tube. The CIS includes a control mechanism similar to current bronchoscopes which allows for flexion of the tip and overall flexibility of the stylet. In contrast to the bronchoscope, however, the CIS includes no fiberoptics or associated components, such as a light source or eyepiece, making the CIS much less expensive to produce.

### CROSS-REFERENCE TO RELATED APPLICATION

The present application is a U.S. nonprovisional patent application of, and claims priority under 35 U.S.C. §119(e) to, U.S. provisional patent application Ser. No. 61/157,547, filed Mar. 4, 2009, which provisional patent application is hereby incorporated herein by reference. The disclosure of the provisional application is contained in Appendix A hereof, which disclosure is hereby incorporated herein by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

The present invention relates to medical apparatus used in the field of anesthesiology and to methods of use of such apparatus for intubation. More particularly, the invention relates to apparatus for atraumatic intubation.

Provision of anesthesia to patients during surgical procedures requires ensuring adequate respiratory function. This is most often accomplished by use of an endotracheal tube (“ETT”). ETTs generally comprise flexible breathing conduits constructed of medical grade plastics that are adapted to be placed in the patient's trachea. The proximate end of the ETT has a standard fitting allowing connection to a source of pressurized gas such as oxygen and anesthetic gases. The distal end is open to deliver these gases to the trachea and lungs of the patient. The distal end usually has a side hole to aid in equal ventilation of both lungs should the ETT be inaccurately positioned. This side hole is not intended to, nor does it, decrease ventilatory resistance in an ETT that is correctly positioned. The ETT typically has an inflatable bladder or balloon which can be inflated once the ETT is in place within the trachea. This seals the trachea allowing positive pressure ventilation to the lungs and protecting them from secretions and gastric contents. During the intubation process the ETT traverses the mouth, pharynx, larynx, and trachea of the patient An exemplary ETT 100 is illustrated in FIG. 1. Another exemplary ETT is disclosed, for example, in U.S. Pat. No. 6,978,784, which is incorporated herein by reference.

Ideally, the ETT is placed in the correct position within the trachea without causing damage to bodily structures. Various devices are available to facilitate such placement. Placement of the ETT may be performed under direct visualization using a laryngoscope. Generally, a laryngoscope consists of a handle (usually incorporating two batteries) and an interchangeable blade with a light source. The handle usually accommodates a power source for the light source and may include, for example, batteries. Laryngoscopes are well known and disclosed, for example, in U.S. Pat. Nos. 4,565,187; 5,095,888; 5,498,231; and 7,153,260, each of which is incorporated herein by reference.

An intubating stylet may be used in conjunction with the laryngoscope for facilitating correct positioning of the ETT. Intubating stylets are well known and disclosed, for example, in U.S. Pat. No. 5,259,377; and U.S. Patent Appl. Publication No. 2007/0287961, each of which is incorporated herein by reference. Intubating stylets may even include a bright light at the distal end for illumination. Unfortunately, however, such stylets generally only provide indirect transcutaneous illumination of the trachea. Direct visualization is not possible when using lighted stylets.

Furthermore, fiberoptic bronchoscopes (“FOBs”)—also sometimes referred to as “flexible bronchoscopes”—may be used with ETTs as an intubating stylets, which FOBs advantageously provide direct visualization. Such use is preferred when a difficult airway is presented, especially when the glottic opening cannot be fully visualized using a laryngoscope. In this respect, a “difficult” intubation is defined by the American Society of Anesthesiologists (“ASA”) as multiple attempts to achieve successful intubation, and it is believed that over one million difficult intubations utilizing the above described laryngoscopes occur each year.

FOBs are well known and disclosed, for example, in U.S. Pat. No. 4,683,874 and U.S. Patent Appl. Publication No. 2005/0272971, which is incorporated herein by reference. An exemplary FOB 200 also is illustrated in FIG. 2. Use of a FOB in conjunction with a laryngoscope for intubation is disclosed, for example, in U.S. Pat. No. 5,776,052, which is incorporated herein by reference.

In general, a FOB includes three basic components: a handpiece, which includes an eyepiece for viewing; an elongated shaft housing fiberoptic bundles, channels, and control wires; and a distal tip containing optics. The FOB also generally includes a cable mechanism, or other mechanism, that is connected to a control mechanisms of the handpiece, and that extends to the distal tip. The cable mechanism enables both flexion and extension of the distal tip by adjustment of a lever or other control of the control mechanism, thereby facilitating navigation of the distal end of the FOB into individual lobe or segment bronchi. FOBs also may include a channel for suctioning or instrumentation, and a high intensity light source, light from which is transmitted through the fiberoptic bundles. The fiberoptic bundles transmit the light to the distal tip where it is used to illuminate the object to be viewed. The optics located in the distal tip transmit the image through another fiberoptic bundle to the handpiece, where the image can be manually viewed with the eye.

A closer view of the handpiece 300 of an exemplary FOB is illustrated in FIG. 3. The handpiece includes a working channel 320, a flexion lever 322, a focusing ring 324, suction 326, and an umbilical cord 328.

FIGS. 4A and 4B illustrate the flexion of the distal tip 310 within a plane, which flexion is controlled by manual movement of the lever of the handpiece 300 illustrated in FIG. 3. Moreover, the plane of flexion may be varied by rotation of the handpiece 300.

When using a FOB with an ETT as an intubating stylet, an appropriate size ETT for the patient is chosen and threaded onto the proximal shaft of the FOB. The tip is of the ETT is lubricated with a water soluble medical lubricant. Importantly, use of an FOB as an intubating stylet allows visualization of the upper airway structures and visual confirmation that the trachea has been entered.

Specifically, the upper airways are traversed with the distal tip of the FOB and the laryngeal structures are visualized and identified. The distal tip of the FOB is advanced through the vocal cords and into the trachea. Once entrance of the FOB into the trachea is visually confirmed, the ETT is slid down the shaft of the FOB, using the FOB as an intubating stylet. The tip of the ETT must traverse the larynx prior to entering the trachea, and it is at this point resistance and obstruction to advancement is frequently encountered. Thereafter the ETT must be positioned accurately within the trachea and is done so either by direct visualization of the bronchi and carina (the first division of the trachea) through the FOB, or by using predetermined norms for ETT position and listening to sounds of breathing. Once positioned and the tracheal balloon inflated, the FOB is removed from the ETT. The proximal end of the ETT is then connected to a pressurized gas source and the patient is ventilated.

Thus, once the trachea has been entered, the FOB is used to guide placement of the ETT by sliding the ETT over the FOB into the trachea. Once the ETT has been successfully placed in the trachea, the FOB is withdrawn and the patient can be ventilated.

A FOB used with an ETT as an intubating stylet also may be coupled with an electronic display for viewing, omitting or replacing use of the eyepiece of the bronchoscope. Such apparatus also is disclosed and taught in U.S. Pat. No. 5,329,940, which is incorporated herein by reference.

Other FOBs with cameras and/or eyepieces are disclosed, for example, in U.S. Pat. Nos. 3,776,222; 4,742,819; 4,846,153 and 5,363,838, each of which is incorporated by reference herein.

Unfortunately, FOBs are very expensive and require significant time to clean and recondition for use. Moreover, FOBs are generally fragile and prone to breaking. Accordingly, while FOBs are believed to be preferred for endotracheal intubation, FOBs are generally used only for difficult airways and in emergency cases, and only when available.

In contrast to such limited use of FOBs, the need to quickly and safely intubate without trauma cannot be overstated. Delayed or failed intubation, and trauma from ETT placement, can cause hypoxic brain injury; hemodynamic instability/stress; bleeding, swelling, laryngospasm, patient discomfort and hoarseness; and even death resulting from complications of the foregoing. Furthermore, if the tracheal tube is not inserted far enough past the vocal cords, the tube may become dislodged and prove to be ineffective in supporting adequate artificial ventilation. The ETT also may inadvertently end up in the esophagus. Esophageal intubations, resulting from either dislodgement or incorrect initial placement have led to severe morbidity and even death. At the other extreme, if inserted too far and beyond the carina, the tube may only permit ventilation of one lung (as opposed to both lungs). Thus, correct tube placement is essential in order to properly ventilate the patient.

Proper intubation requires positioning the tip of the tracheal tube within the trachea, midway between the patient's vocal cords and carina. As mentioned above, while direct laryngoscopy can be sufficient in many cases to intubate a patient, such procedure but does not permit precise confirmation of tip location or tracheal inspection, and use of such procedure includes the risk of delay when difficult airways are encountered and a FOB must be located or obtained for performing the intubation.

As will be appreciated from the foregoing description, a need exists for apparatus and methods for facilitating safer and more efficient endotracheal intubation.

Video laryngoscopes represent a recent advancement in the specific art of laryngoscopes, and in the general field of anesthesiology, which addresses such need. A video laryngoscope is a laryngoscope designed to visualize the larynx using video technology. The video laryngoscope uses a video camera mounted on a scope, whereas previous laryngoscopes (also sometimes referred to as “direct” laryngoscopes) use a rigid scope with a light on the blade or intra-oral portion and requires a direct line of sight to the upper airway structures including the larynx and tracheal opening.

As with the direct laryngoscopes described above, the video laryngoscope is used to facilitate placement of the ETT, and such placement may be performed with an intubating stylet or FOB used as an intubating stylet. Moreover, when an FOB is used as an intubating stylet, the imaging functionality of the FOB may not be used, as the video laryngoscope provides the direct visualization for proper placement of the ETT. Use of the FOB with the video laryngoscope is preferred over other intubating stylets because the FOB is believed to provide greater control and navigation during intubation because of the ability to flex and extend the distal tip. However, such use for FOBs is limited due to the generally limited use and availability of FOBs described above.

A well known and widely received video laryngoscope is sold under the trademark GLIDESCOPE, and uses a CCD or CMOS camera placed at a point on the blade, back from the tip, designed to provide perspective of the field in front of the camera. A 60 degree angle of the blade provides an ability to see around the corner behind the tongue to an anteriorly located larynx. This 60 degree angulation reduces the need to displace the tongue for a direct line of sight to the larynx. It is believed that video laryngoscopes significantly increase the safety of and efficiency in endotracheal intubation, and their use will continue to enjoy increased adoption by practitioners.

An exemplary GLIDESCOPE video laryngoscope 500 is illustrated in FIG. 5. Other video laryngoscopes are known, and include the “McGrath” video laryngoscope; the Storz C-Mac; Pentax AWS; and Berci DCI video laryngoscopes. These laryngoscopes use a variety of form factors, including in some instances placement of a monitor on the handle, and/or channels to attempt to guide the endotracheal tube into the trachea.

The GLIDESCOPE COBALT video laryngoscope is a variant of the GLIDESCOPE apparatus that has a reusable video camera with light emitting core which has a disposable or single use external shell for prevention of cross infection. This permits decreased time in reconditioning and preparing the GLIDESCOPE COBALT video laryngoscope after use.

Video laryngoscopes are also well disclosed in the patent literature including, for example, U.S. Pat. Nos. 6,354,993; 6,652,453; and 6,676,598, each of which is incorporated herein by reference.

While video laryngoscopes are an important advancement that addresses the need for facilitating safer and more efficient endotracheal intubation, it is believed that such need may be further addressed through yet additional improvements and enhancements in the general field of anesthesiology. One or more embodiments of the present invention are believed to address such continuing need. Indeed, one or more preferred embodiments of the present invention are believed to advantageously provide the benefits of the combined use of a video laryngoscope and FOB as an intubating stylet without the acquisition and maintenance costs and other disadvantages associated with FOBs that lead to their limited availability and use in practice.

### SUMMARY OF THE INVENTION

The present invention includes many aspects and features. Moreover, while many aspects and features relate to, and are described in, the context of anesthesiology, the present invention is not limited to use only in anesthesiology, as will become apparent from the following summaries and detailed descriptions of aspects, features, and one or more embodiments of the present invention.

Accordingly, one aspect of the present invention includes the combination of a video laryngoscope and a controllable intubating stylet (hereinafter “CIS”) that is flexible and similar in construction to a FOB, but that does not include the fiberoptics and associated components of the FOB, including the light source and eyepiece. Importantly, the flexible stylet does include the control mechanism and cable mechanism found in the FOB—or another mechanism for providing flexion and extension of the distal tip of the stylet. In a feature, the CIS includes therein a conduit or lumen for local anesthetic injection for awake intubation, for the introduction of oxygen, or for suction.

In another aspect of the invention, a method of intubating a trachea of a patient includes the steps of: providing a controllable intubating stylet (CIS), the CIS comprising a flexible stylet and a control mechanism that controls flexion of a tip of the flexible stylet, the CIS including no fiberoptics; providing a video laryngoscope; displaying a view of an opening of the trachea of the patient using the video laryngoscope; inserting the CIS into an endotracheal tube; directing the CIS into the trachea through the displayed opening; maneuvering the endotracheal tube over the CIS and into the trachea through the displayed opening; and thereafter, removing the CIS from the trachea for oxygenating and ventilating of the patient via the endotracheal tube.

In a feature thereof, the CIS that is provided does not comprise an eyepiece, does not comprise a light source, and does not comprise a camera.

In another feature, the CIS that is provided includes a lumen for local anesthetic injection for awake intubation, for the introduction of oxygen, or for suction.

In a feature, the control mechanism of the CIS that is provided is structurally the same as that of a fiberoptic bronchoscope used for controlling flexion of the tip of the fiberoptic bronchoscope.

In a feature, the CIS that is provided is shorter in length than a fiberoptic bronchoscope.

In a feature, the CIS that is provided includes a stylet having a length of around 50 centimeters.

In a feature, the CIS that is provided includes a stylet having a length of around 25-30 centimeters.

In a feature, the CIS that is provided is not suitable for entry into the lungs.

In a feature, the video laryngoscope comprises a video GLIDESCOPE.

In a feature, the CIS that is provided further comprises a handpiece and a handle for rotation of the handpiece and subsequent controlled, manual variation of a plane of flexion of the tip of the flexible stylet, and wherein the method further comprises manipulating the handle about an axis of the handpiece while causing the tip of the flexible style to undergo flexion and extension within a flexion plane, and advancing and withdrawing the stylet relative to the tracheal opening while manipulating the handle about the axis of the handpiece. Preferably, the manipulating, advancing and withdrawing are performed by a single person.

In another aspect, a controllable intubating stylet includes: a flexible stylet and a control mechanism that controls flexion of a tip of the flexible stylet; and a lumen. The controllable intubating stylet does not comprise any fiberoptics, does not comprise an eyepiece, does not comprise a light source, and does not comprise a camera.

In a feature, the lumen provides for application of local anesthetic injection for awake intubation.

In a feature, the lumen provides for introduction of oxygen.

In a feature, the lumen provides for the application of suction to an area of the trachea.

In a feature, the mechanism is that of a fiberoptic bronchoscope used for controlling flexion of the tip of the fiberoptic bronchoscope.

In a feature, the control mechanism comprises a thumb control for controlled, manual flexing of the tip of the flexible stylet.

In a feature, the control mechanism comprises a multi-direction toggle control for controlling flexion of the distal tip in any plane about the longitudinal axis of the CIS at the distal end thereof.

In a feature, the control mechanism comprises two controls by each of which the distal tip is flexed and extended in a controlled manner in a respective orthogonal plane.

In a feature, the mechanism is that of an endoscope designed for colonoscopies.

In another aspect, a combination for use by anesthesia and other health care providers to achieve successful intubation of the trachea for general anesthesia as well as other medical conditions that require control of the airway includes a controllable intubating stylet (CIS) and a video laryngoscope. The video laryngoscope is used to visualize the tracheal opening. The CIS is inserted into an endotracheal tube and directed into the trachea, and the endotracheal tube is then be maneuvered over the stylet into the trachea and the CIS is removed, whereby the patient can then be oxygenated and ventilated via the endotracheal tube.

In addition to the aforementioned aspects and features of the present invention, it should be noted that the present invention further encompasses the various possible combinations and subcombinations of such aspects and features. Moreover, aspects and features of the present invention include methods of using apparatus in accordance with each of the foregoing aspects and features thereof.

### BRIEF DESCRIPTION OF THE DRAWINGS

One or more preferred embodiments of the present invention now will be described in detail with reference to the accompanying drawings, wherein:

FIG. 1 illustrates an exemplary ETT.

FIG. 2 illustrates an exemplary FOB.

FIG. 3 illustrates a closer view of a handpiece of an exemplary FOB.

FIGS. 4A-4B illustrate the flexion of a distal tip of a FOB within a plane, which flexion is controlled by various movement of the lever of the handpiece (seen in FIG. 3).

FIG. 5 illustrates an exemplary GLIDESCOPE video laryngoscope.

Each of FIGS. 6-7 illustrate an exemplary GLIDESCOPE video laryngoscope, wherein the illustrated GLIDESCOPE video laryngoscope is shown in use for visualization of the upper airway structures including the larynx and tracheal opening.

FIG. 8 illustrates a first controllable intubating stylet in accordance with a preferred embodiment of the present invention.

FIG. 9 illustrates a second controllable intubating stylet in accordance with a preferred embodiment of the present invention.

FIG. 10 illustrates a third controllable intubating stylet in accordance with a preferred embodiment of the present invention.

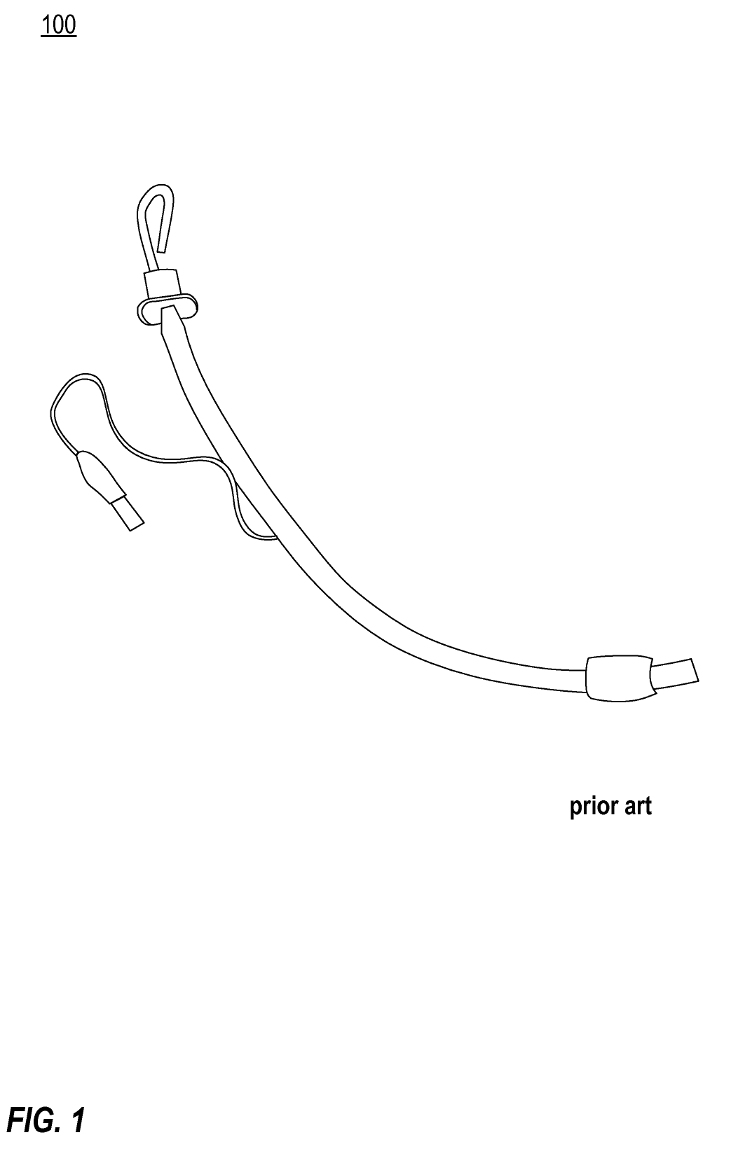

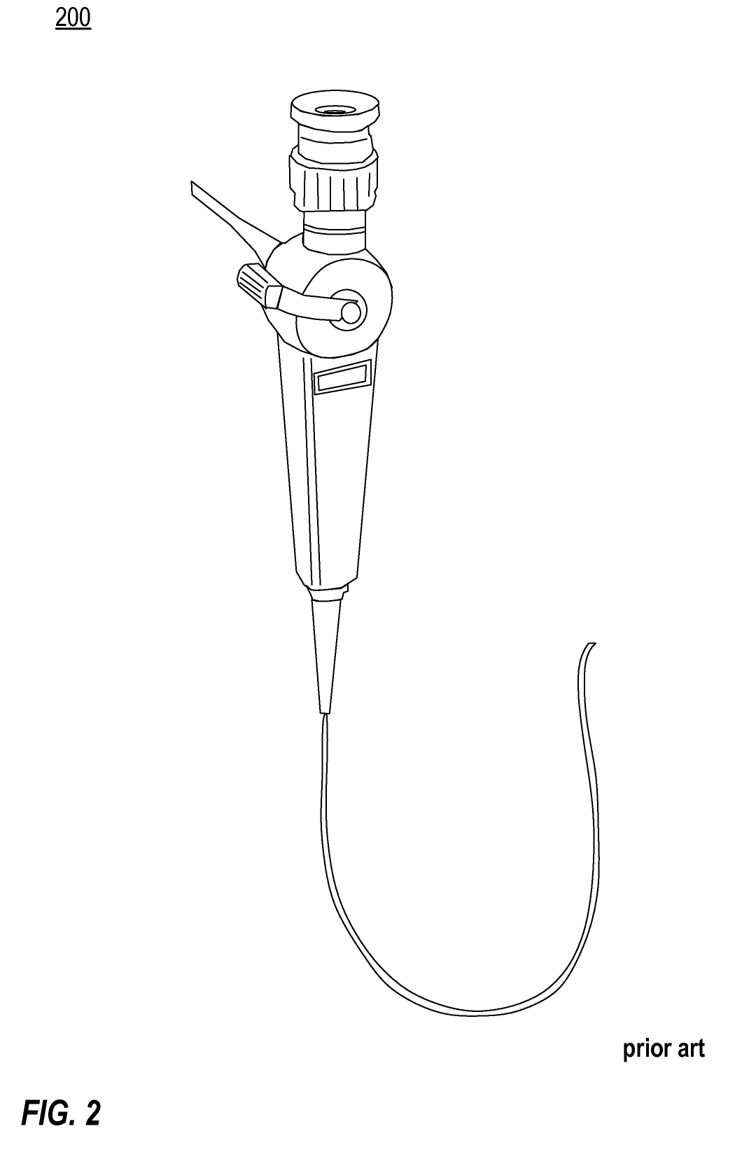

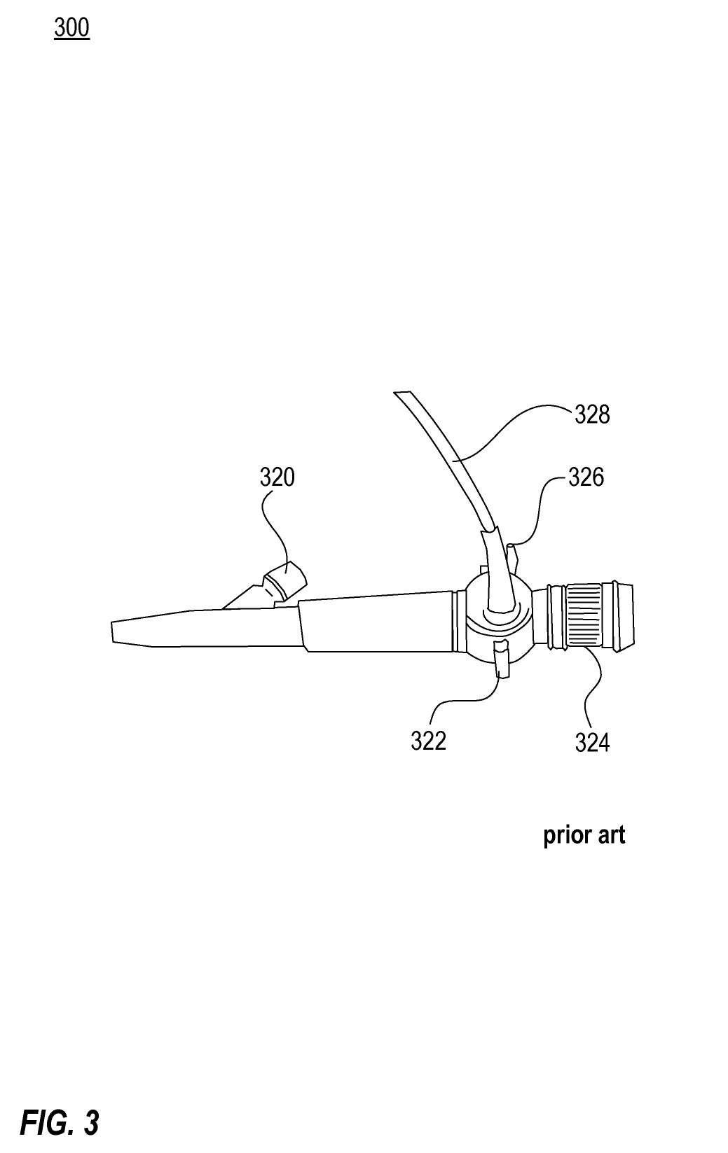

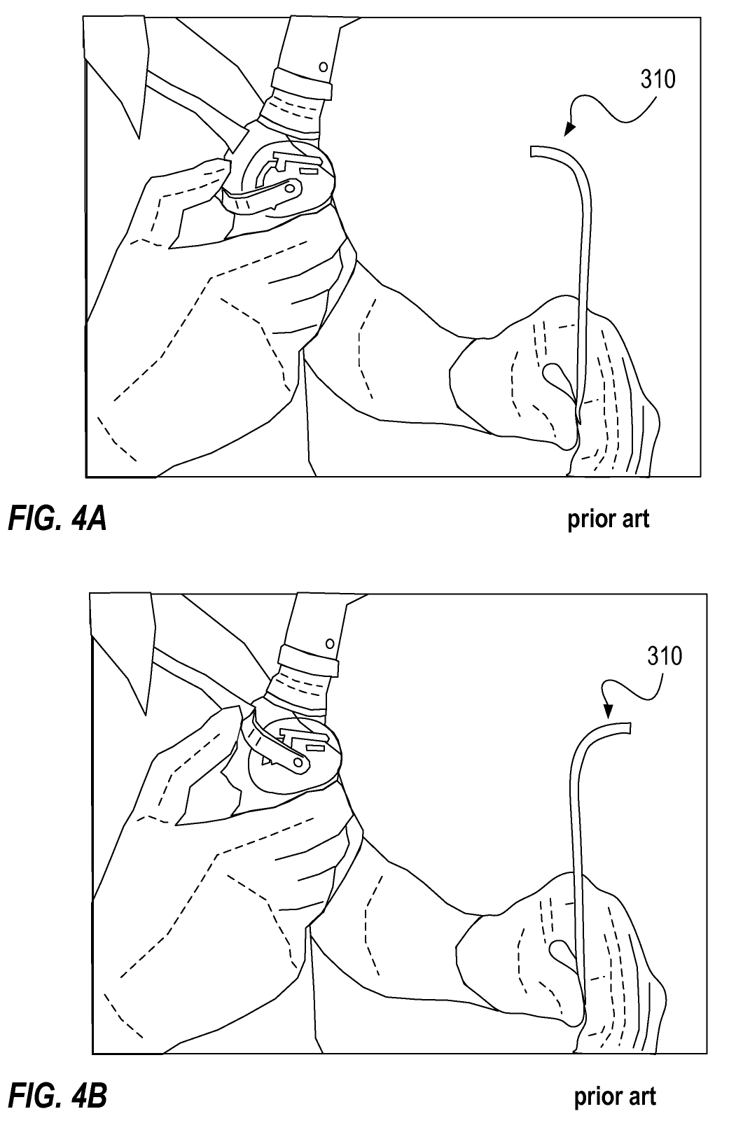

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, one or more preferred embodiments of the present invention are next described. The following description of one or more preferred embodiments is merely exemplary in nature and is in no way intended to limit the invention, its implementations, or uses.

A combination in accordance with a preferred embodiment of the present invention includes a video laryngoscope and, in particular, a GLIDESCOPE video laryngoscope such as that shown in FIGS. 6 and 7, used in combination with a controllable intubating stylet (hereinafter “CIS”) that is flexible and similar in construction to a FOB, but that does not include the fiberoptics and associated components of the FOB, including the light source and eyepiece. In contrast, the flexible stylet does include the control mechanism and cable mechanism found in the FOB—or another mechanism for providing flexion and extension of the distal tip of the stylet.

An example of a control mechanism and cable mechanism that may be utilized also is believed to be disclosed in one or more of the references incorporated herein by reference. Furthermore, the CIS preferable is shorter in length than the FOB, and preferably is between about 25 centimeters and 50 centimeters. In particular, the CIS preferably is developed with varied lengths to include long stylets (50 cm; including use for double lumen endotracheal tubes); and regular stylets (25-30 cm). The CIS preferably is developed with small diameter stylets for pediatric cases. Unlike a FOB, the CIS need not be suitable for entry into the lungs.

Notably, it is believed that such a CIS would be substantially less expensive than a FOB due to the omission of the fiberoptics and associated components, yet still provide the navigational benefits of the FOB for use as an intubating stylet. Moreover, it is believed that the CIS is easy to clean and, therefore, enjoys a significantly reduced downtime for reconditioning after use. The CIS is also believed to be more durable than a FOB, and not as easily breakable due to the absence of the fiberoptics and components associated therewith, including light source and eyepiece. Indeed, whereas a FOB might cost around $15,000, it is believed that a CIS would cost around $300 and, therefore, may be priced for acquisition for use, for example, in every operating room of a hospital. In contrast, a single FOB customarily might be shared among the various operating rooms of a hospital.

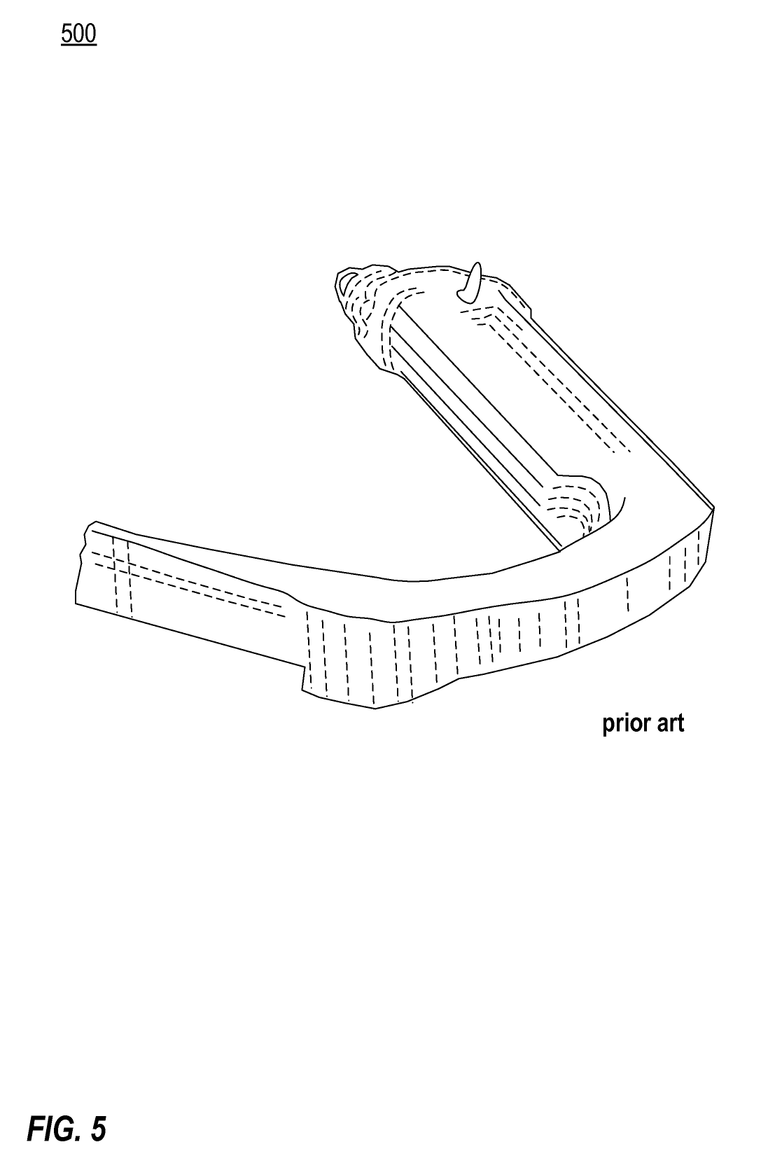

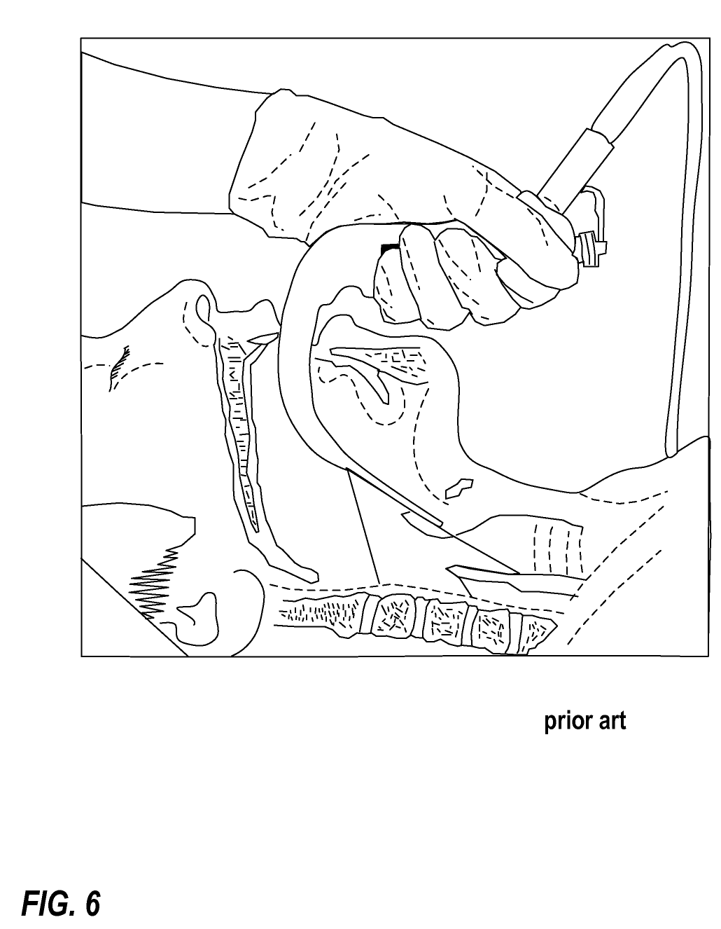

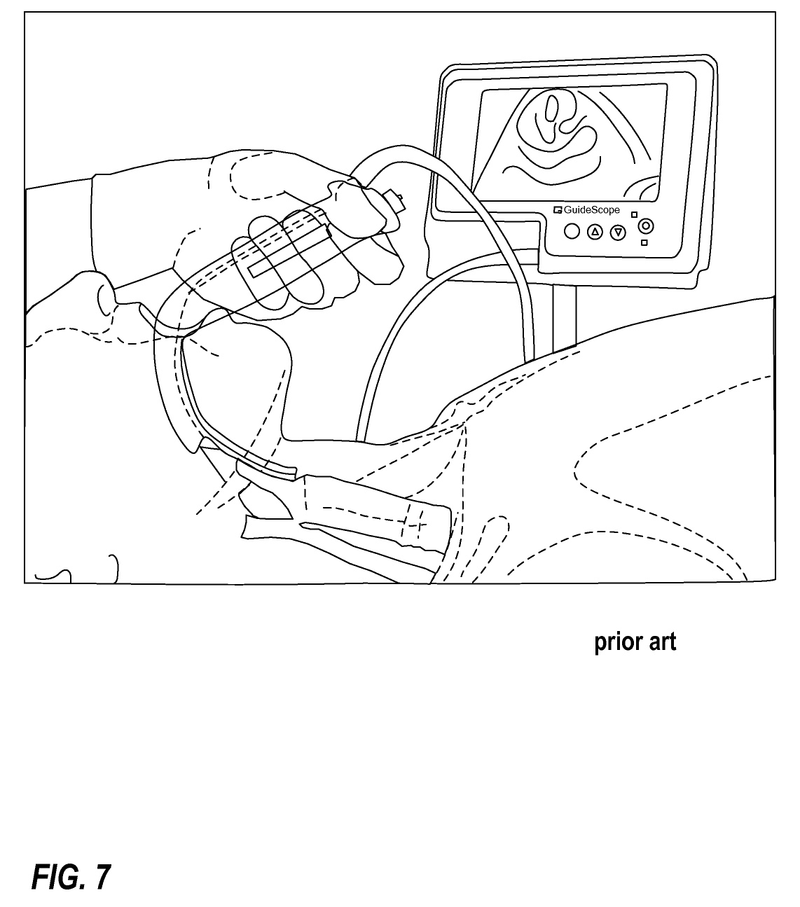

An exemplary GLIDESCOPE video laryngoscope for use with the CIS is represented in FIG. 5 as well as in each of FIGS. 6-7, wherein the GLIDESCOPE video laryngoscope is illustrated in use for visualization of the upper airway structures including the larynx and tracheal opening.

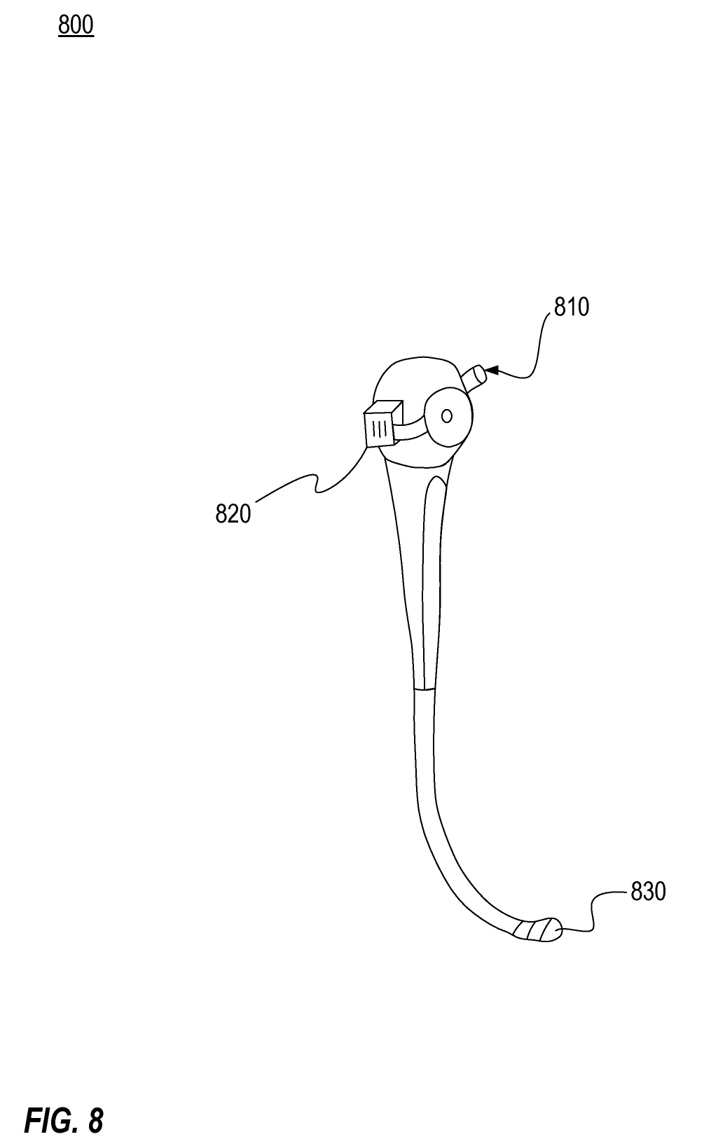

A first CIS 800 in accordance with a preferred embodiment of the invention is illustrated in FIG. 8. The CIS 800 optionally includes therein a conduit or lumen 810 for local anesthetic injection for awake intubation, for the introduction of oxygen, or for suction (e.g. a port for local anesthesia, oxygen, or suction). The CIS 800 also includes a thumb control 820 for controlled, manual flexing of the distal tip 830.

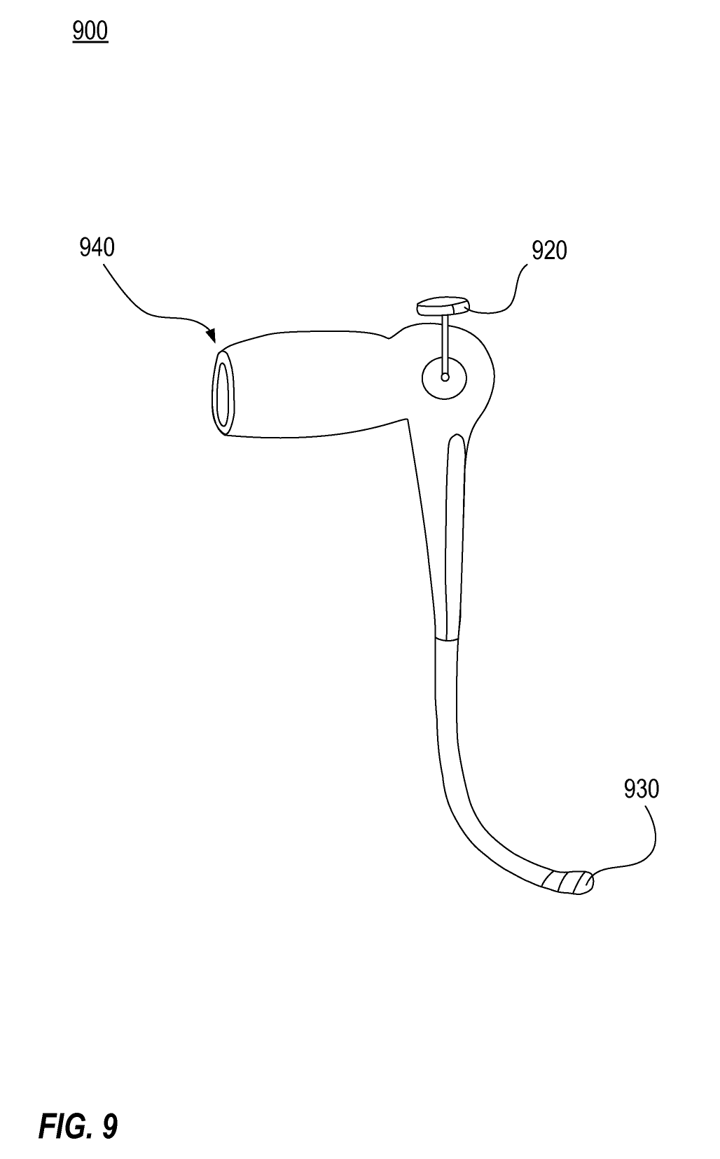

A second CIS 900 in accordance with a preferred embodiment of the invention is illustrated in FIG. 9. Like the CIS 800, the CIS 900 includes a thumb control 920 for controlled, manual flexing of the distal, flexible tip 930 within a plane of flexion. The CIS 900 further includes, however, a handle 940 for control of rotation of the handpiece and for subsequent controlled, manual variation of the plane of flexion of the flexible tip 930. It is also believed that the handle 940 enables a two-handed (and one person) procedure. In this respect, the handle 940 allows the CIS to be manipulated right and left about an axis of the handpiece thereof while the thumb control will allow for flexion and extension within a flexion plane. Furthermore, the entire unit can be advanced or withdrawn, as needed, by the handle 940. The CIS 900 is not shown with a conduit or lumen for local anesthetic injection for awake intubation, for the introduction of oxygen, or for suction; however, such a conduit of lumen may optionally be included.

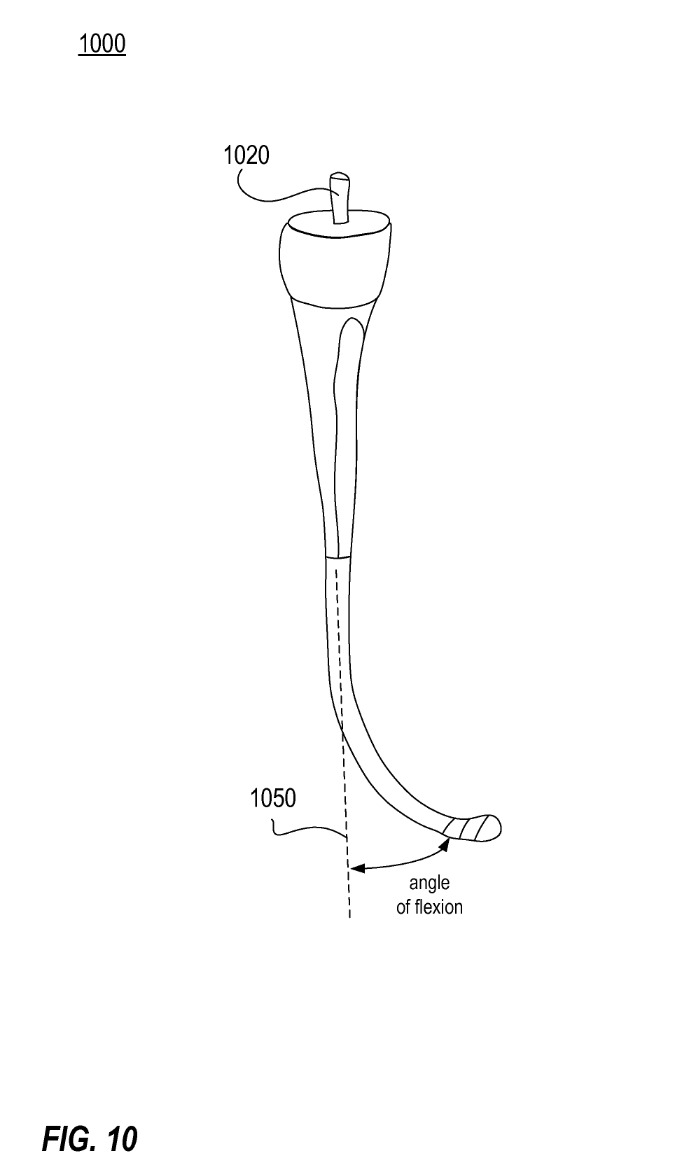

A third CIS 1000 in accordance with a preferred embodiment of the invention is illustrated in FIG. 10. The CIS 1000 includes a multi-direction toggle control 1020 for controlling flexion of the distal tip in any plane about the longitudinal axis 1050 of the CIS 1000 at the distal end thereof.

An additional CIS in accordance with a preferred embodiment of the invention (not shown) includes two controls by each of which the distal tip is flexed and extended in a controlled manner in respective orthogonal planes. Such control mechanisms and cables are believed to be well known and currently used in endoscopes designed for colonoscopies.

In methods of use of the foregoing CIS disclosed herein, and in accordance with preferred embodiments of the invention, a video laryngoscope is used to visualize the tracheal opening, and the CIS is inserted into an endotracheal tube and directed into the trachea. The endotracheal tube then is maneuvered over the CIS and into the trachea, and thereafter, the CIS is removed. The patient can then be oxygenated and ventilated by way of the endotracheal tube. The CIS includes a control mechanism similar to current FOBs that permits flexion of the tip and overall flexibility and control of the CIS during insertion of the tip into the trachea. Furthermore, at least some preferred methods of use of a CIS in accordance with one or more preferred embodiments of the invention may be accomplished by a single person using two hands.

It is believed that use of the video laryngoscope with a CIS in accordance with the invention will achieve successful intubation in nearly all cases.

Furthermore, advantages to utilizing the CIS over a fiberoptic bronchoscope are believed to include:
Durability: fiberoptic bronchoscopes are fragile and require storage in a large full length box often attached to a “difficult airway cart”.
Accessibility: CIS's can be purchased for every operating room or hooked to the side of every GLIDESCOPE video laryngoscope.
Decreased cost: CIS's can be manufactured at a fraction of the cost of a bronchoscope.
Decreased maintenance: fiberoptic bronchoscopes require a lengthy cleaning process prior to using them on different patients. It often takes over 20 minutes to return a bronchoscope into circulation. The CIS can be wiped clean and reused quickly.

Based on the foregoing description, it will be appreciated by those persons skilled in the art that a controllable intubating stylet for use by anesthesia and other health care providers has been disclosed herein, as well as combination and use thereof. In contrast to the bronchoscope, however, the CIS includes no fiberoptics or associated components, such as a light source or eyepiece, making the CIS much less expensive to produce than bronchoscopes.

It will further be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. A method of intubating a trachea of a patient, comprising:
(a) providing a controllable intubating stylet (CIS), the CIS comprising a flexible stylet and a control mechanism that controls flexion of a tip of the flexible stylet, the CIS including no fiberoptics;
(b) providing a video laryngoscope;
(c) displaying a view of an opening of the trachea of the patient using the video laryngoscope;
(d) inserting the CIS into an endotracheal tube;
(e) directing the CIS into the trachea through the displayed opening;
(f) maneuvering the endotracheal tube over the CIS and into the trachea through the displayed opening; and
(g) thereafter, removing the CIS from the trachea for oxygenating and ventilating of the patient via the endotracheal tube.

2. The method of claim 1, wherein the CIS that is provided does not comprise an eyepiece, does not comprise a light source, and does not comprise a camera.

3. The method of claim 1, wherein the CIS that is provided includes a lumen for local anesthetic injection for awake intubation, for the introduction of oxygen, or for suction.

4. The method of claim 1, wherein the control mechanism of the CIS that is provided is structurally the same as that of a fiberoptic bronchoscope used for controlling flexion of the tip of the fiberoptic bronchoscope.

5. The method of claim 1, wherein the CIS that is provided is shorter in length than a fiberoptic bronchoscope.

6. The method of claim 1, wherein the CIS that is provided includes a stylet having a length of around 50 centimeters.

7. The method of claim 1, wherein the CIS that is provided includes a stylet having a length of around 25-30 centimeters.

8. The method of claim 1, wherein the CIS that is provided is not suitable for entry into the lungs.

9. The method of claim 1, wherein the video laryngoscope comprises a video GLIDESCOPE.

10. The method of claim 1, wherein the CIS that is provided further comprises a handpiece and a handle for rotation of the handpiece and subsequent controlled, manual variation of a plane of flexion of the tip of the flexible stylet, and wherein the method further comprises manipulating the handle about an axis of the handpiece while causing the tip of the flexible style to undergo flexion and extension within a flexion plane, and advancing and withdrawing the stylet relative to the tracheal opening while manipulating the handle about the axis of the handpiece.

11. The method of claim 10, wherein said manipulating and said advancing and withdrawing are performed by a single person.

12. A controllable intubating stylet comprising:
(a) a flexible stylet and a control mechanism that controls flexion of a tip of the flexible stylet; and
(b) a lumen;
(c) wherein the controllable intubating stylet does not comprise any fiberoptics, does not comprise an eyepiece, does not comprise a light source, and does not comprise a camera.

13. The controllable intubating stylet of claim 12, wherein the lumen provides for application of local anesthetic injection for awake intubation.

14. The controllable intubating stylet of claim 12, wherein the lumen provides for introduction of oxygen.

15. The controllable intubating stylet of claim 12, wherein the lumen provides for the application of suction to an area of the trachea.

16. The controllable intubating stylet of claim 12, wherein the mechanism is that of a fiberoptic bronchoscope used for controlling flexion of the tip of the fiberoptic bronchoscope.

17. The controllable intubating stylet of claim 12, wherein the control mechanism comprises a thumb control for controlled, manual flexing of the tip of the flexible stylet.

18. The controllable intubating stylet of claim 12, wherein the control mechanism comprises a multi-direction toggle control for controlling flexion of the distal tip in any plane about the longitudinal axis of the CIS at the distal end thereof.

19. The controllable intubating stylet of claim 12, wherein the control mechanism comprises two controls by each of which the distal tip is flexed and extended in a controlled manner in a respective orthogonal plane.

20. The controllable intubating stylet of claim 19, wherein the mechanism is that of an endoscope designed for colonoscopies.
